(19) United States
(12) Patent Application Publication
(10) Pub. No.: US 2026/0058003 A1
(43) Pub. Date: Feb. 26, 2026

(54) SYSTEM AND METHOD FOR GENERATING DENOISED SPECTRAL CT IMAGES FROM SPECTRAL CT IMAGE DATA ACQUIRED USING A SPECTRAL CT IMAGING SYSTEM

(71) Applicant: GE Precision Healthcare LLC, Waukesha, WI (US)

(72) Inventors: Alma Eguizabal, Stockholm (SE); Mats Persson, Västerhaninge (SE); Dennis Hein, Stockholm (SE)

(21) Appl. No.: 19/102,792

(22) PCT Filed: Aug. 10, 2023
(86) PCT No.: PCT/US2023/072028

§ 371 Date: Feb. 10, 2025

Related U.S. Application Data
(60) Provisional application No. 63/396,686, filed on Aug. 10, 2022.

(51) Int. Cl. G16H 30/40; G06T 5/60; G06T 5/70; G16H 30/20

(52) U.S. Cl. CPC G16H 30/40; G06T 5/60; G06T 5/70; G16H 30/20; G06T 2207/10081; G06T 2207/20084

(57) ABSTRACT

Various systems and methods are provided for denoising spectral CT image data, the system and method comprising determining a denoised linear estimation of spectral CT image data by maximizing or minimizing a first objective function, wherein at least one parameter of the denoised linear estimation is determined by at least one machine learning system. The denoiser is based on a Linear Minimum Mean Square Error (LMMSE) estimator. The LMMSE is very fast to compute, but not commonly used for CT image denoising, due to its inability to adapt the amount of denoising to different parts of the image and the difficulty to derive accurate statistical properties from the CT image data. To overcome these problems, a model-based deep learning model, such as a deep neural network that preserves a model based LMMSE structure.

### CROSS-REFERENCE TO RELATED APPLICATIONS

This is a national stage application under 35 U.S.C. § 371 (c) of PCT Application No. PCT/US2023/072028, filed on Aug. 10, 2023, which claims priority to U.S. Provisional Application No. 63/396,686, filed on Aug. 10, 2022, the disclosures of which are incorporated herein by reference in its entirety.

### BACKGROUND

Embodiments of the subject matter disclosed herein relate to X-ray technology and X-ray imaging and corresponding imaging reconstruction and imaging tasks. In particular, the embodiments of the subject matter disclosed herein relate to a system and method for generating denoised spectral computed tomography (CT) images from spectral CT image data acquired using a spectral (energy-resolving) CT imaging system.

Radiographic imaging systems such as computed tomography (CT) imaging systems and other more general X-ray imaging systems have been used for years in medical applications, such as for medical diagnostics and treatment.

Normally, an X-ray imaging system such as a CT imaging system includes an X-ray source and an X-ray detector consisting of multiple detector modules comprising one or many detector elements, for independent measuring of X-ray intensities. The X-ray source emits X-rays, which pass through a subject or object to be imaged and are then received by the detector. The energy spectrum of a typical medical X-ray tube is broad and ranges from zero up to 160 keV. The X-ray detector therefore typically detects X-rays with varying energy levels.

The X-ray source and X-ray detector are typically arranged to rotate on a rotating member of a gantry, around the subject or object. The emitted X-rays are attenuated by the subject or object as they pass through, and the resulting transmitted X-rays are measured by the detector. The measured data may then be used to reconstruct images of the subject or object.

A challenge for X-ray detectors is to extract maximum information from the detected X-rays to provide input to an image of an object or subject where the object or subject is depicted in terms of density, composition, and structure.

It may be useful with a brief overview of an illustrative general X-ray imaging system according to the prior art with reference to FIG. 1A. In this illustrative example the X-ray imaging system 100 comprises an X-ray source 10, an X-ray detector 20 and an associated image processing system 30. In general, the X-ray detector 20 is configured to register radiation from the X-ray source 10, which optionally has been focused by optional X-ray optics or collimators and passed through an object, a subject or a part thereof. The X-ray detector 20 is connectable to the image processing system 30 via suitable read-out electronics, which is at least partly integrated in the X-ray detector 20, to enable image processing and/or image reconstruction by the image processing system 30.

By way of example, a conventional CT imaging system includes an X-ray source and an X-ray detector arranged in such a way that projection images of the subject or object can be acquired in different viewing angles covering at least 180 degrees. This is most commonly achieved by mounting the source and detector on a support, e.g., a rotating member of a gantry, that is able to rotate around the subject or object. An image containing the projections registered in the different detector elements for the different view angles is called a sinogram. In the following, a collection of projections registered in the different detector elements for different view angles will be referred to as a sinogram even if the detector is two-dimensional, making the sinogram a three-dimensional image.

FIG. 1B is a schematic diagram illustrating an example of an X-ray imaging system setup according to the prior art, showing projection lines from an X-ray source through an object to an X-ray detector.

A further development of X-ray imaging is energy-resolved X-ray imaging, also known as spectral X-ray imaging, where the X-ray transmission is measured for several different energy levels. This can be achieved by letting the source switch rapidly between two different emission spectra, by using two or more X-ray sources emitting different X-ray spectra, or by using an energy-discriminating detector which measures the incoming radiation in two or more energy levels. An example of such a detector is a multi-bin photon counting detector, where each registered photon generates a current pulse which is compared to a set of thresholds, thereby counting the number of photons incident in each of a number of energy bins.

A spectral X-ray projection measurement results in a projection image for each energy level. A weighted sum of these projection images can be made to optimize the contrast-to-noise ratio (CNR) for a specified imaging task as described in “SNR and DQE analysis of broad spectrum X-ray imaging”, Tapiovaara and Wagner, Phys. Med. Biol. 30, 519.

Another technique enabled by energy-resolved X-ray imaging is basis material decomposition. This technique utilizes the fact that all substances built up from elements with low atomic number, such as human tissue, have linear attenuation coefficients whose energy dependence can be expressed, to a good approximation, as a linear combination of two (or more) basis functions:

$$μ ( E ) = {a}_{1} {f}_{1} ( E ) + {a}_{2} {f}_{2} ( E )$$

where f₁ and f₂ are basis functions and a₁ and a₂ are the corresponding basis coefficients. More, generally, fᵢ are basis functions and aᵢ are corresponding basis coefficients, where i=1, . . . , N where N is the total number of basis functions. If there is one or more element in the imaged volume with high atomic number, high enough for a K-absorption edge to be present in the energy range used for the imaging, one basis function must be added for each such element. In the field of medical imaging, such K-edge elements can typically be iodine or gadolinium, substances that are used as contrast agents.

Basis material decomposition has been described in “Energy-selective reconstructions in X-ray computerized tomography”, Alvarez, Macovski, Phys Med Biol. 1976; 21(5):733-744. In basis material decomposition, the integral of each of the basis coefficients, Aᵢ= [symbol] aᵢdl for i=1, . . . , N where N is the number of basis functions, is inferred from the measured data in each projection ray [symbol] from the source to a detector element. In one implementation, this is accomplished by first expressing the expected registered number of counts in each energy bin as a function of Aᵢ:

$${λ}_{i} = {\int }_{E = 0}^{∞} {S}_{i} ( E ) exp ( - {\sum }_{j = 1}^{N} {A}_{j} {f}_{j} ( E ) ) dE$$

where λᵢ is the expected number of counts in energy bin i, E is the energy, Sᵢ is a response function which depends on the spectrum shape incident on the imaged object, the quantum efficiency of the detector and the sensitivity of energy bin i to X-rays with energy E. Even though the term “energy bin” is most commonly used for photon counting detectors, this formula can also describe other energy resolving X-ray imaging systems such as multi-layer detectors, kVp switching X-ray sources or multiple X-ray source systems.

Then, the maximum likelihood method may be used to estimate Aᵢ, under the assumption that the number of counts in each bin is a Poisson distributed random variable. This is accomplished by minimizing the negative log-likelihood function, e.g., see “K-edge imaging in X-ray computed tomography using multi-bin photon counting detectors”, Roessl and Proksa, Phys. Med. Biol. 52(2007), 4679-4696:

$${Â}_{1} , … , {Â}_{N} = {arg min}_{{A}_{1} , … , {A}_{N}} {\sum }_{i = 1}^{{M}_{b}} {λ}_{i} ( {A}_{1} , … , {A}_{N} ) - {m}_{i} {In λ}_{i} ( {A}_{1} , … , {A}_{N} )$$

where mᵢ is the number of measured counts in energy bin i and M_{b} is the number of energy bins.

When the resulting estimated basis coefficient line integral Âᵢ for each projection line is arranged into an image matrix, the result is a material specific projection image, also called a basis image, for each basis i. This basis image can either be viewed directly (e.g., in projection X-ray imaging) or taken as input to a reconstruction algorithm to form maps of basis coefficients aᵢ inside the object (e.g., in CT imaging). In either case, the result of a basis decomposition can be regarded as one or more basis image representations, such as the basis coefficient line integrals or the basis coefficients themselves.

Standard data management procedures for X-ray imaging systems present different approaches to optimize data acquisition, but possibly at a cost of e.g., spatial resolution, noise level and/or system complexity.

A map of basis coefficients aᵢ inside an object is referred to as a basis material image, a basis image, a material image, a material-specific image, a material map or a basis map.

However, a well-known limitation of this technique is that the variance of the estimated line integrals normally increases with the number of bases used in the basis decomposition. Among other things, this results in an unfortunate trade-off between improved tissue quantification and increased image noise.

Further, accurate basis decomposition with more than two basis functions may be hard to perform in practice, and may result in artifacts, bias or excessive noise. Such a basis decomposition may also require extensive calibration measurements and data preprocessing to yield accurate results.

Due to the inherent complexity in many image reconstruction tasks, artificial intelligence (AI) and deep learning have started to being used in general image reconstruction with satisfactory results. It would be desirable to be able to use AI and deep learning for X-ray imaging tasks including spectral CT. However, there is in general a need for improved denoising methods in spectral CT.

Another current problem in deep learning image reconstruction is its limited explainability. An image may seemingly look like it has a very low noise level but in reality, contains errors due to biases in the neural network estimator. An explainable AI technique would be able to provide some information about why the output image has particular characteristics, based on the input image and the training data.

Another drawback with existing AI techniques is that they are typically not tunable and thus only provide a single output image for a given input image, meaning that there is no possibility to adjust the characteristics of the output image without retraining the network.

Accordingly, there is a need for improved denoising methods for spectral computed tomography (CT) in general, and a need for denoising methods with a degree of explainability and tunability in particular.

### SUMMARY

This summary introduces concepts that are described in more detail in the detailed description. It should not be used to identify essential features of the claimed subject matter, nor to limit the scope of the claimed subject matter.

The inventors have appreciated that image reconstruction in spectral CT imaging is more challenging for two main reasons: 1) the multiple bins and materials in the analysis, as well as the improved resolution, increase the amount of processing data considerably; and 2) efficient material decomposition and image reconstruction methods tend to generate noisy images that do not satisfy expected image quality. Thus, there is a need for denoising the material images.

In the present disclosure, a fast denoiser is presented, which is based on deep learning and a Linear Minimum Mean Square Error (LMMSE), combined in a model-based deep learning approach. In this way, the prior knowledge is incorporated into the denoiser, but preserving a linear estimator structure in order to provide interpretability to the result. The architecture with a linear estimator with a matrix and a vector estimated with a deep neural network provides a large amount of flexibility that can outperform conventional deep learning denoisers.

This interpretability allows images with desired properties to be generated by adjusting the coefficients of the estimated linear estimator. For example, one or more coefficients in the linear estimator can be increased or decreased in order to decrease large area bias or improve preservation of fine details in the image.

This adjustment can for example be done in advance of image reconstruction, e.g., when developing a reconstruction method, or in real time while displaying an image to the end user, allowing the user to adjust the image to obtain the desired image properties.

According to a first aspect, there is provided a method for denoising spectral CT image data comprising determining a denoised linear estimation of spectral CT image data by maximizing or minimizing a first objective function, wherein at least one parameter of the denoised linear estimation is determined by at least one machine learning system.

According to a second aspect there is provided a CT imaging system comprising an X-ray source configured to emit X-rays; an X-ray detector configured to generate spectral CT image data; and a processor configured to determine a denoised linear estimation of the generated spectral CT image data based on maximizing or minimizing a first objective function, wherein the processor is further configured to determine at least one parameter of the linear estimation by at least one machine learning system.

Various systems and methods are provided for denoising spectral CT image data, the system and method comprising determining a denoised linear estimation of spectral CT image data by maximizing or minimizing a first objective function, wherein at least one parameter of the denoised linear estimation is determined by at least one machine learning system. The denoiser is based on a Linear Minimum Mean Square Error (LMMSE) estimator. The LMMSE is very fast to compute, but not commonly used for CT image denoising, due to its inability to adapt the amount of denoising to different parts of the image and the difficulty to derive accurate statistical properties from the CT image data. To overcome these problems, a model-based deep learning model, such as a deep neural network that preserves a model based LMMSE structure.

### BRIEF DESCRIPTION OF DRAWINGS

The embodiments, together with further objects and advantages thereof, may best be understood by making reference to the following description taken together with the accompanying drawings, in which:

FIGS. 1A and 1B are schematic diagrams illustrating an example X-ray imaging system.

FIG. 2 is a schematic diagram illustrating another example of an X-ray imaging system, such as a CT imaging system.

FIG. 3 is a schematic block diagram of a CT imaging system as an illustrative example of an X-ray imaging system.

FIG. 4 is a schematic diagram illustrating another example of relevant parts of an X-ray imaging system, such as a CT imaging system.

FIG. 5 is a schematic illustration of a photon counting circuit and/or device according to an exemplary embodiment.

FIG. 6 is a schematic diagram illustrating an example of a semiconductor detector sub-module according to an exemplary embodiment.

FIG. 7 is a schematic diagram illustrating an example of semiconductor detector sub-module according to another exemplary embodiment.

FIG. 8A is a schematic diagram illustrating an example of a semiconductor detector sub-module according to yet another exemplary embodiment.

FIG. 8B is a schematic diagram illustrating an example of a set of tiled detector sub-modules, where each detector sub-module is a depth-segmented detector sub-module and Application Specific Integrated Circuits (ASICs) or corresponding circuitry are arranged below the detector sub-modules as seen from the direction of the incoming X-rays.

FIG. 9 shows a schematic representation of a conventional convolutional neural network (CNN) based denoising technique, in which a CNN maps directly a noise image to a clean image.

FIG. 10 shows a schematic representation of the presented denoising technique, in which a CNN is used to map linear model parameters W and b that are used to generate the clean image.

FIG. 11 shows an example of the interpretability of the proposed model, with three parameters, σ, β and λ used to control the different components of an output image.

FIG. 12 shows an example of interpretation of what the two components in a=Wx+b provide to the result.

FIG. 13 is an empirical demonstration of the parallelism of the technique and an LMMSE: first row represents the estimator components of a regular learned Linear Minimum Mean Square Error (LMMSE) for a particular phantom example, and second row their equivalent with our learned approach.

FIG. 14 is a schematic diagram illustrating an example of a computer implementation according to an embodiment.

### DETAILED DESCRIPTION

Embodiments of the present disclosure will now be described, by way of example, with reference to the figures.

For a better understanding, it may be useful to continue with an introductory description of non-limiting examples of an overall X-ray imaging system in which data processing and transferring according to the inventive concept may be implemented.

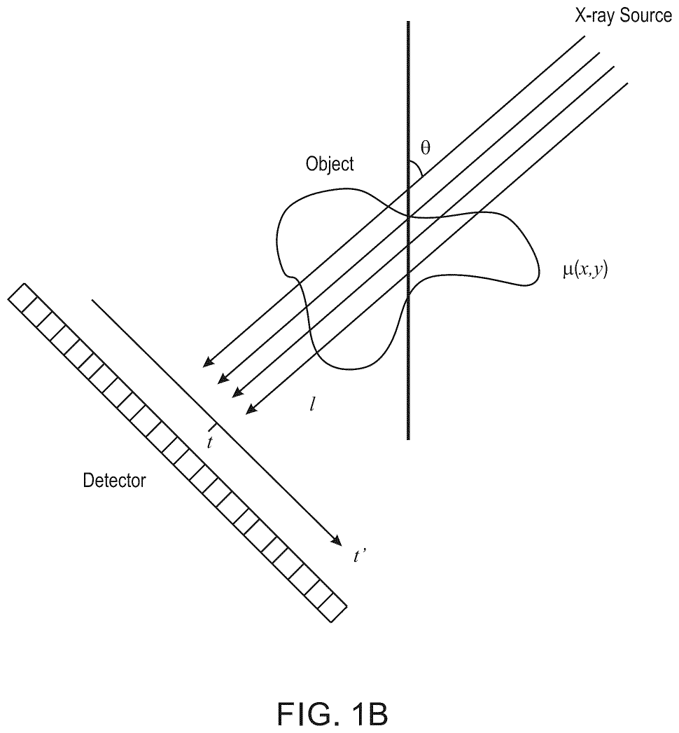

FIG. 2 is a schematic diagram illustrating an example of an X-ray imaging system 100, such as a CT imaging system, comprising an X-ray source 10, which emits X-rays, an X-ray detector 20 with an X-ray detector, which detects X-rays after they have passed through the object, analog processing circuitry 25, which processes the raw electrical signals from the X-ray detector and digitizes it, digital processing circuitry 40, which may carry out further processing operations on the measured data, such as applying corrections, storing it temporarily, or filtering, and a computer 50, which stores the processed data and may perform further post-processing and/or image reconstruction. The digital processing circuitry 40 may comprise a digital processor. According to an exemplary embodiment, all or part of the analog processing circuitry 25 may be implemented in the X-ray detector 20. The X-ray source and X-ray detector may be coupled to a rotating member of a gantry 11 of the CT imaging system 100.

The overall X-ray detector may be regarded as the X-ray detector system 20, or the X-ray detector 20 combined with the associated analog processing circuitry 25.

In communication with and electrically coupled to the analog processing circuitry 25 is an image processing system 30, which may include digital processing circuitry 40 and/or a computer 50, which may be configured to perform image reconstruction based on the image data from the X-ray detector. The image processing system 30 may, thus, be seen as the computer 50, or alternatively the combined system of the digital processing circuitry 40 and the computer 50, or possibly the digital processing circuitry 40 by itself if the digital processing circuitry is further specialized also for image processing and/or reconstruction.

An example of a commonly used X-ray imaging system is a CT imaging system, which may include an X-ray source or X-ray tube that produces a fan beam or cone beam of X-rays and an opposing array of X-ray detectors measuring the fraction of X-rays that are transmitted through a patient or object. The X-ray source or X-ray tube and X-ray detector are mounted in a gantry 11 that can rotate around the imaged object.

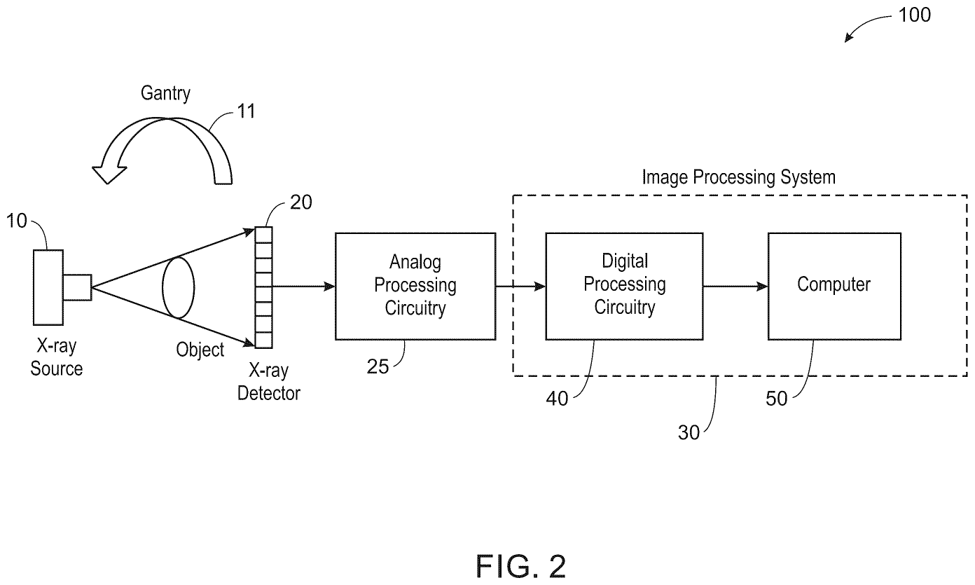

FIG. 3 schematically shows a CT imaging system 100 as an illustrative example of an X-ray imaging system. The CT imaging system comprises a computer 50 receiving commands and scanning parameters from an operator via an operator console 60 that may have a display 62 and some form of operator interface, e.g., a keyboard, mouse, joy stick, touch screen or other input device. The operator supplied commands and parameters are then used by the computer 50 to provide control signals to an X-ray controller 41, a gantry controller 42 and a table controller 43. To be specific, the X-ray controller 41 provides power and timing signals to the x-ray source 10 to control emission of X-rays onto the object or patient lying on the table 12. The gantry controller 42 controls the rotating speed and position of the gantry 11 comprising the X-ray source 10 and the X-ray detector 20. By way of example, the X-ray detector 20 may be a photon counting X-ray detector. The table controller 43 controls and determines the position of the patient table 12 and the scanning coverage of the patient. There is also a detector controller 44, which is configured for controlling and/or receiving data from the X-ray detector 20.

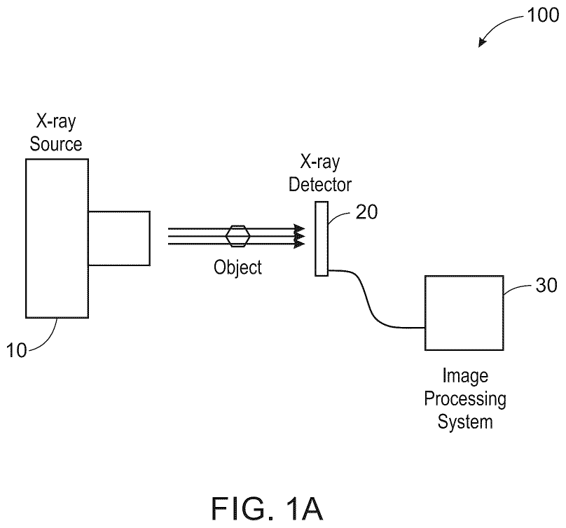

In an embodiment, the computer 50 also performs post-processing and image reconstruction of the image data output from the X-ray detector 20. The computer 50 thereby corresponds to the image processing system 30 as shown in FIGS. 1 and 2. The associated display 62 allows the operator to observe the reconstructed images and other data from the computer 50.

The X-ray source 10 arranged in the gantry 11 emits X-rays. An X-ray detector 20, which may be in the form of a photon counting X-ray detector, detects the X-rays after they have passed through the object or patient. The X-ray detector 20 may for example be formed by plurality of pixels, also referred to as sensors or detector elements, and associated processing circuitry, such as Application Specific Integrated Circuits (ASICs), arranged in detector modules. A portion of the analog processing may be implemented in the pixels, whereas any remaining processing is implemented in, for instance, the ASICs. In an embodiment, the processing circuitry (ASICs) digitizes the analog signals from the pixels. The processing circuitry (ASICs) may also comprise a digital processing, which may carry out further processing operations on the measured data, such as applying corrections, storing it temporarily, and/or filtering. During a scan to acquire X-ray projection data, the gantry and the components mounted thereon rotate about an isocenter 13.

Modern X-ray detectors normally need to convert the incident X-rays into electrons, this typically takes place through the photoelectric effect or through Compton interaction and the resulting electrons are usually creating secondary visible light until its energy is lost and this light is in turn detected by a photo-sensitive material. There are also detectors, which are based on semiconductors and in this case the electrons created by the X-ray are creating electric charge in terms of electron-hole pairs which are collected through an applied electric field.

There are detectors operating in an energy integrating mode in the sense that they provide an integrated signal from a multitude of X-rays. The output signal is proportional to the total energy deposited by the detected X-rays.

X-ray detectors with photon counting and energy resolving capabilities are becoming common for medical X-ray applications. The photon counting detectors have an advantage since in principle the energy for each X-ray can be measured which yields additional information about the composition of the object. This information can be used to increase the image quality and/or to decrease the radiation dose.

Generally, a photon counting X-ray detector determines the energy of a photon by comparing the height of the electric pulse generated by a photon interaction in the detector material to a set of comparator voltages. These comparator voltages are also referred to as energy thresholds. Generally, the analog voltage in a comparator is set by a digital-to-analog converter (DAC). The DAC converts a digital setting sent by a controller to an analog voltage to which the heights of the photon pulses can be compared.

A photon counting detector counts the number of photons that have interacted in the detector during a measurement time. A new photon is generally identified by the fact that the height of the electric pulse exceeds the comparator voltage of at least one comparator. When a photon is identified, the event is stored by incrementing a digital counter associated with the channel.

When using several different threshold values, an energy-discriminating photon counting detector is obtained, in which the detected photons can be sorted into energy bins corresponding to the various threshold values. Sometimes, this type of photon counting detector is also referred to as a multi-bin detector. In general, the energy information allows for new kinds of images to be created, where new information is available and image artifacts inherent to conventional technology can be removed. In other words, for an energy-discriminating photon counting detector, the pulse heights are compared to a number N of programmable thresholds (T1-TN) in the comparators and are classified according to pulse-height, which in turn is proportional to energy. In other words, a photon counting detector comprising more than one comparator is here referred to as a multi-bin photon counting detector. In the case of multi-bin photon counting detector, the photon counts are stored in a set of counters, typically one for each energy threshold. For example, one count can be assigned to the highest energy threshold that the photon pulse has exceeded. In another example, counters keep track of the number of times that the photon pulse cross each energy threshold.

As an example, edge-on is a special, non-limiting design for a photon counting detector, where the X-ray sensors such as X-ray detector elements or pixels are oriented edge-on to incoming X-rays.

For example, such photon counting detectors may have pixels in at least two directions, wherein one of the directions of the edge-on photon counting detector has a component in the direction of the X-rays. Such an edge-on photon counting detector is sometimes referred to as a depth-segmented photon counting detector, having two or more depth segments of pixels in the direction of the incoming X-rays. It should be noted that one detector element may correspond to one pixel, and/or a plurality of detector elements corresponds to one pixel and/or the data signal from a plurality of detector elements may be used for one pixel.

Alternatively, the pixels may be arranged as an array (non-depth-segmented) in a direction substantially orthogonal to the direction of the incident X-rays, and each of the pixels may be oriented edge-on to the incident X-rays. In other words, the photon counting detector may be non-depth-segmented, while still arranged edge-on to the incoming X-rays.

By arranging the edge-on photon counting detector edge-on, the absorption efficiency can be increased, in which case the absorption depth can be chosen to any length, and the edge-on photon counting detector can still be fully depleted without going to very high voltages.

A conventional mechanism to detect X-ray photons through a direct semiconductor detector basically works as follows. The energy of the X-ray interactions in the detector material are converted to electron-hole pairs inside the semiconductor detector, where the number of electron-hole pairs is generally proportional to the photon energy. The electrons and holes are drifted towards the detector electrodes and backside (or vice versa). During this drift, the electrons and holes induce an electrical current in the electrode, a current which may be measured.

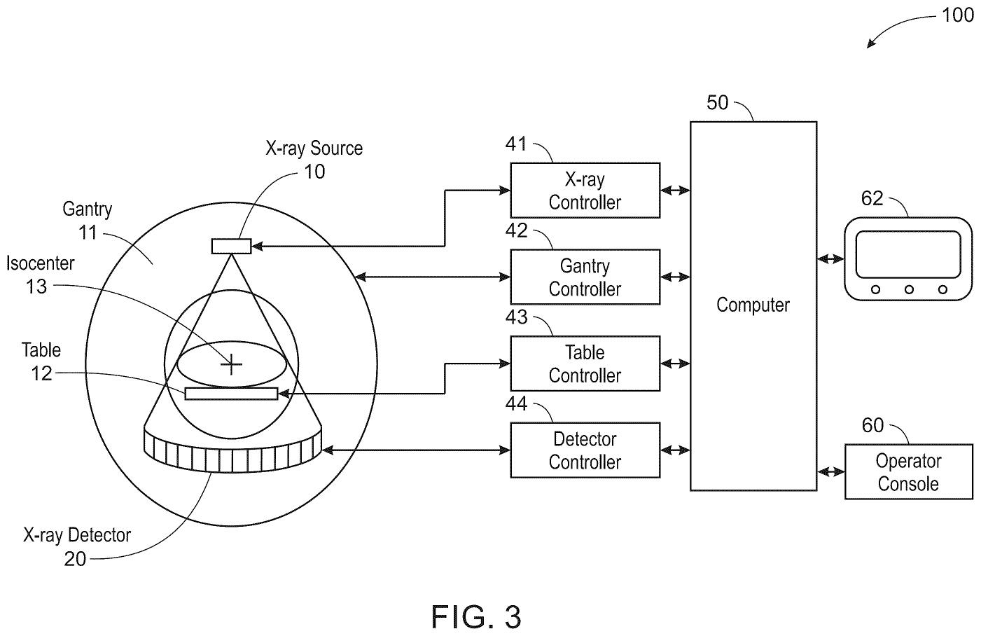

As illustrated in FIG. 4, signal(s) is/are routed via routing paths 26 from detector elements 22 of the X-ray detector to inputs of analog processing circuitry (e.g., ASICs) 25. It should be understood that the term Application Specific Integrated Circuit (ASIC) is to be interpreted broadly as any general circuit used and configured for a specific application. The ASIC processes the electric charge generated from each X-ray and converts it to digital data, which can be used to obtain measurement data such as a photon count and/or estimated energy. The ASICs are configured for connection to digital processing circuitry so the digital data may be sent to digital processing circuitry 40 and/or one or more memory circuits or components 45 and finally the data will be the input for image processing circuitry 30 or computer 50 in FIG. 2 to generate a reconstructed image.

As the number of electrons and holes from one X-ray event is proportional to the energy of the X-ray photon, the total charge in one induced current pulse is proportional to this energy. After a filtering step in the ASIC, the pulse amplitude is proportional to the total charge in the current pulse, and therefore proportional to the X-ray energy. The pulse amplitude can then be measured by comparing its value with one or more thresholds (THR) in one or more comparators (COMP), and counters are introduced by which the number of cases when a pulse is larger than the threshold value may be recorded. In this way it is possible to count and/or record the number of X-ray photons with an energy exceeding an energy corresponding to respective threshold value (THR) which has been detected within a certain time frame.

The ASIC typically samples the analog photon pulse once every Clock Cycle and registers the output of the comparators. The comparator(s) (threshold) outputs a one or a zero depending on whether the analog signal was above or below the comparator voltage. The available information at each sample is, for example, a one or a zero for each comparator representing weather the comparator has been triggered (photon pulse was higher than the threshold) or not.

In a photon counting detector, there is typically a Photon Counting Logic which determines if a new photon has been registered and, registers the photons in counter(s). In the case of a multi-bin photon counting detector, there are typically several counters, for example one for each comparator, and the photon counts are registered in the counters in accordance with an estimate of the photon energy. The logic can be implemented in several different ways. Two of the most common categories of Photon Counting Logic are the non-paralyzable counting modes, and the paralyzable counting modes. Other photon counting logics include, for example, local maxima detection, which counts, and possibly also registers the pulse height of, detected local maxima in the voltage pulse.

There are many benefits of photon counting detectors including, but not limited to: high spatial resolution; less sensitivity to electronic noise; good energy resolution; and material separation capability (spectral imaging ability). However, energy integrating detectors have the advantage of high count-rate tolerance. The count-rate tolerance comes from the fact/recognition that, since the total energy of the photons is measured, adding one additional photon will always increase the output signal (within reasonable limits), regardless of the amount of photons that are currently being registered by the detector. This advantage is one of the main reasons that energy integrating detectors are the standard for medical CT today.

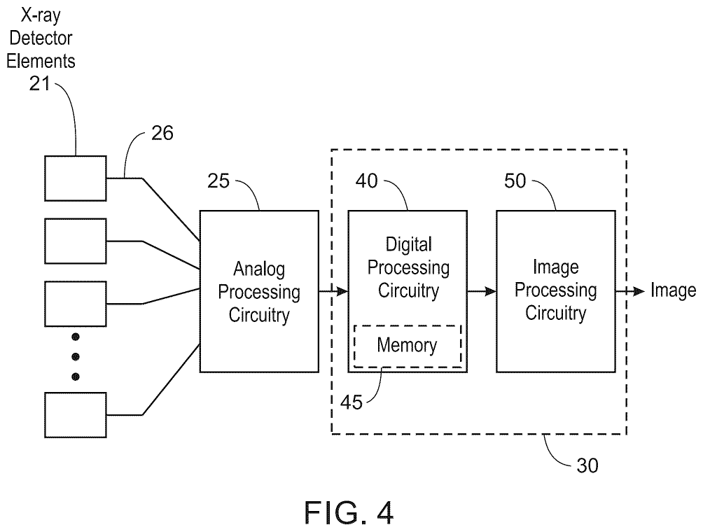

FIG. 5 shows a schematic illustration of a photon counting circuit and/or device according to an exemplary embodiment.

When a photon interacts in a semiconductor material, a cloud of electron-hole pairs is created. By applying an electric field over the detector material, the charge carriers are collected by electrodes attached to the detector material. The signal is routed from the detector elements to inputs of parallel processing circuits, e.g., ASICs. In one example, the ASIC can process the electric charge such that a voltage pulse is produced with maximum height proportional to the amount of energy deposited by the photon in the detector material.

The ASIC may include a set of comparators 302 where each comparator 302 compares the magnitude of the voltage pulse to a reference voltage. The comparator output is typically zero or one (0/1) depending on which of the two compared voltages that is larger. Here we will assume that the comparator output is one (1) if the voltage pulse is higher than the reference voltage, and zero (0) if the reference voltage is higher than the voltage pulse. Digital-to-analog converters (DACs), 301 can be used to convert digital settings, which may be supplied by the user or a control program, to reference voltages that can be used by the comparators 302. If the height of the voltage pulse exceeds the reference voltage of a specific comparator, we will refer to the comparator as triggered. Each comparator is generally associated with a digital counter 303, which is incremented based on the comparator output in accordance with the photon counting logic.

As previously mentioned, when the resulting estimated basis coefficient line integral Âᵢ for each projection line is arranged into an image matrix, the result is a material specific projection image, also called a basis image, for each basis i. This basis image can either be viewed directly (e.g., in projection X-ray imaging) or taken as input to a reconstruction algorithm to form maps of basis coefficients aᵢ inside the object (e.g., in CT). Anyway, the result of a basis decomposition can be regarded as one or more basis image representations, such as the basis coefficient line integrals or the basis coefficients themselves.

It will be appreciated that the mechanisms and arrangements described herein can be implemented, combined and re-arranged in a variety of ways.

For example, embodiments may be implemented in hardware, or at least partly in software for execution by suitable processing circuitry, or a combination thereof.

The steps, functions, procedures, and/or blocks described herein may be implemented in hardware using any conventional technology, such as discrete circuit or integrated circuit technology, including both general-purpose electronic circuitry and application-specific circuitry.

Alternatively, or as a complement, at least some of the steps, functions, procedures, and/or blocks described herein may be implemented in software such as a computer program for execution by suitable processing circuitry such as one or more processors or processing units.

In the following, non-limiting examples of specific detector module implementations will be discussed. More particularly, these examples refer to edge-on oriented detector modules and depth-segmented detector modules. Other types of detectors and detector modules may also be feasible.

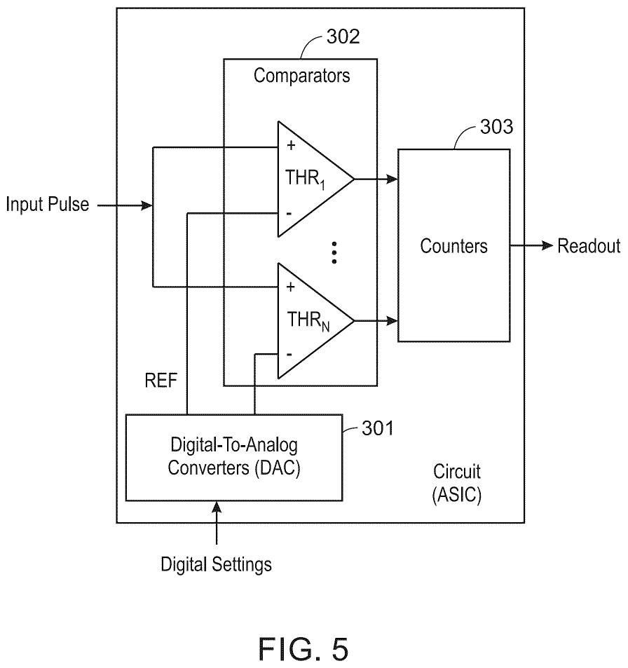

FIG. 6 is a schematic diagram illustrating an example of a semiconductor detector sub-module according to an exemplary embodiment. This is an example of a detector module 21 with a semiconductor sensor having a plurality of detector elements or pixels 22, where each detector element (or pixel) is normally based on a diode having a charge collecting electrode as a key component. The X-rays enter through the edge of the detector module.

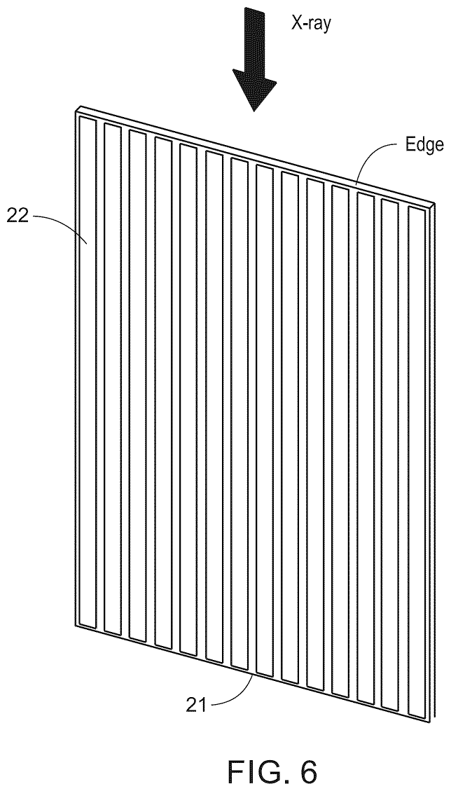

FIG. 7 is a schematic diagram illustrating an example of semiconductor detector sub-module according to another exemplary embodiment. In this example, the detector module 21 with the semiconductor sensor is also split into a plurality of depth segments or detector elements 22 in the depth direction, again assuming the X-rays enter through the edge of the detector module.

Normally, a detector element is an individual X-ray sensitive sub-element of the detector. In general, the photon interaction takes place in a detector element and the thus generated charge is collected by the corresponding electrode of the detector element.

Each detector element typically measures the incident X-ray flux as a sequence of frames. A frame is the measured data during a specified time interval, called frame time.

Depending on the detector topology, a detector element may correspond to a pixel, especially when the detector is a flat-panel detector. A depth-segmented detector may be regarded as having a number of detector strips, each strip having a number of depth segments. For such a depth-segmented detector, each depth segment may be regarded as an individual detector element, especially if each of the depth segments is associated with its own individual charge collecting electrode.

The detector strips of a depth-segmented detector normally correspond to the pixels of an ordinary flat-panel detector, and therefore sometimes also referred to as pixel strips. However, it is also possible to regard a depth-segmented detector as a three-dimensional pixel array, where each pixel corresponds to an individual depth segment/detector element.

The semiconductor sensors may be implemented as so called Multi-Chip Modules (MCMs) in the sense that the semiconductor sensors are used as base substrates for electric routing and for a number of ASICs which are attached preferably through so called flip-chip technique. The routing will include a connection for the signal from each pixel or detector element to the ASIC input as well as connections from the ASIC to external memory and/or digital data processing. Power to the ASICs may be provided through similar routing taking into account the increase in cross-section which is required for the large currents in these connections, but the power may also be provided through a separate connection. The ASICS may be positioned on the side of the active sensor and this means it can be protected from the incident X-rays if an absorbing cover is placed on top and it can also be protected from scattered X-rays from the side by positioning an absorber also in this direction.

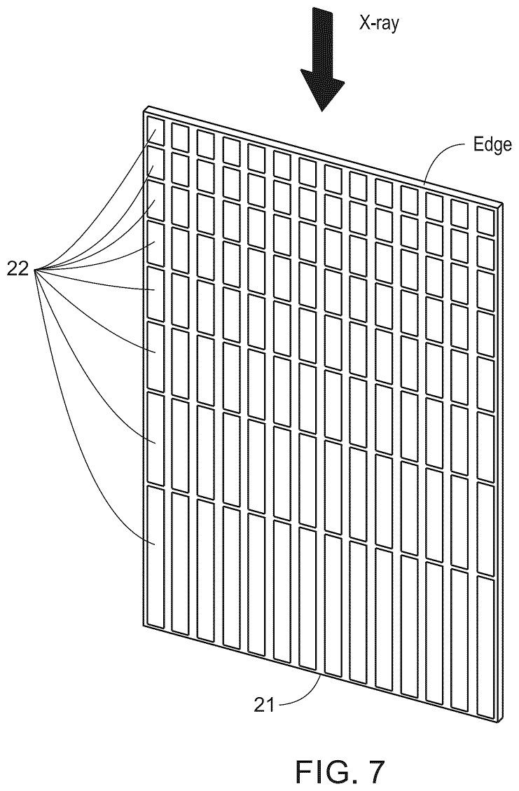

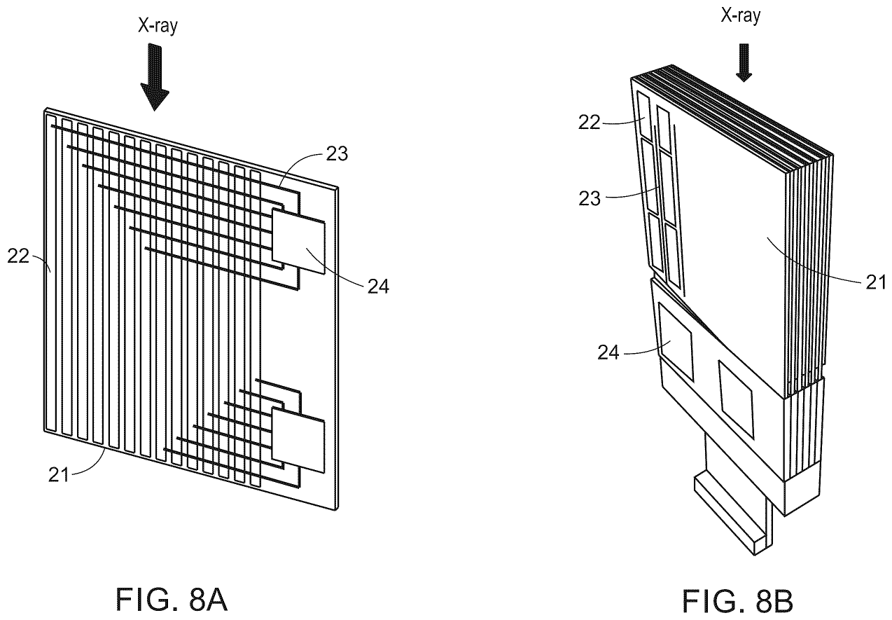

FIG. 8A is a schematic diagram illustrating a detector module implemented as a MCM similar to embodiments in U.S. Pat. No. 8,183,535. In this example, it is illustrated how the semiconductor sensor 21 also can have the function of a substrate in a MCM. The signals are routed by routing paths 23 from the detector elements 22 to inputs of parallel processing circuits 24 (e.g., ASICs) that are positioned next to the active sensor area. The ASICs process the electric charge generated from each X-ray and converts it to digital data which can be used to detect a photon and/or estimate the energy of the photon. The ASICs may have their own digital processing circuitry and memory for small tasks. And, the ASICs may be configured for connection to digital processing circuitry and/or memory circuits or components located outside of the MCM and finally the data will be used as input for reconstructing an image.

However, the employment of depth segments also brings two noticeable challenges to a silicon-based photon counting detector. First, a large number of ASIC channels has to be employed to process data fed from the associated detector segments. In addition to the increased number of channels due to both the smaller pixel size and the depth segmentation, multi-energy bin further increases the data size. Second, since the given X-ray input counts are divided into smaller pixels, segments and energy bins, each bin has much lower signal and so the detector calibration/correction requires more than several orders of magnitude more calibration data to minimize statistical uncertainty.

Naturally, the several orders of magnitude larger data size slow down both data handling and pre-processing in addition to the need of larger computing resources, hard drive, memory, and central processing unit (CPU) or graphics processing unit (GPU). When the size of data is 10 Gigabytes instead of 10 Megabyte, for example, the data handling time, read and write, can take 1000 times longer.

A problem in any counting X-ray photon detector is the pile-up problem. When the flux rate of X-ray photons is high there may be problems in distinguishing between two subsequent charge pulses. As mentioned above, the pulse length after the filter depends on the shaping time. If this pulse length is larger than the time between two X-ray photon induced charge pulses, the pulses will grow together, and the two photons are not distinguishable and may be counted as one pulse. This is called pile-up. One way to avoid pile-up at high photon flux is thus to use a small shaping time, or to use depth-segmentation.

For pileup calibration vector generation, the pileup calibration data needs to be pre-processed for spit correction. For material decomposition vector generation, the material decomposition data should preferably be pre-processed for both spit and pileup correction. For patient scan data, the data needs to be pre-processed for spit, pileup and material decomposition before the image reconstruction ensues. These are simplified examples to explain “pre-processing” since the actual pre-processing steps can include several other calibration steps as needed, like reference normalization and air calibration. The term “processing” may indicate only the final step in each calibration vector generation or patient scan, but it is used interchangeably in some cases.

FIG. 8B is a schematic diagram illustrating an example of a set of tiled detector sub-modules, where each detector sub-module is a depth-segmented detector sub-module and the ASICs or corresponding circuitry 24 are arranged below the detector elements 22 as seen from the direction of the incoming X-rays, allowing for routing paths 23 from the detector elements 22 to the parallel processing circuits 24 (e.g., ASICs) in the space between detector elements.

Artificial Intelligence (AI) and deep learning have started to being used in general image reconstruction with some satisfactory results. However, a current problem in deep-learning image reconstruction is its limited explainability. An image may seemingly look like it has a very low noise level but in reality, contains errors due to biases in the neural network estimator.

In general, deep learning relates to machine learning methods based on artificial neural networks or similar architectures with representation learning. Learning can be supervised, semi-supervised or unsupervised. Deep learning systems such as deep neural networks, deep belief networks, recurrent neural networks and convolutional neural networks have been applied to various technical fields including computer vision, speech recognition, natural language processing, social network filtering, machine translation, and board game programs, where they have produced results comparable to and in some cases surpassing human expert performance.

The adjective “deep” in deep learning originates from the use of multiple layers in the network. Early work showed that a linear perceptron cannot be a universal classifier, and that a network with a non-polynomial activation function with one hidden layer of unbounded width can on the other hand so be. Deep learning is a modern variation which is concerned with an unlimited number of layers of bounded size, which permits practical application and optimized implementation, while retaining theoretical universality under mild conditions. In deep learning the layers are also permitted to be heterogeneous and to deviate widely from biologically informed connectionist models, for the sake of efficiency, trainability, and understandability.

The inventors have realized that there is a need for denoising algorithms with improved performance for spectral CT, and in particular for algorithms with improved explainability.

The proposed technology is generally applicable for providing denoised image data in spectral CT based on neural networks and/or deep learning.

In order to provide an exemplary framework for facilitating the understanding of the proposed technology, a specific example of deep learning-based image reconstruction in the particular context of spectral CT image reconstruction will now be given.

It should though be understood that the proposed technology for providing an indication of the confidence in deep-learning image reconstruction in spectral CT applications is generally applicable to deep-learning based image reconstruction for CT, and not limited to the following specific example of deep-learning based image reconstruction.

The inventors disclose a new and fast denoiser that is based on a Linear Minimum Mean Square Error (LMMSE) estimator. The LMMSE is very fast to compute, but not commonly used for CT image denoising, probably due to its inability to adapt the amount of denoising to different parts of the image and the difficulty to derive accurate statistical properties from the CT data. To overcome these problems the inventors, propose a model-based deep learning strategy, that is, a deep neural network that preserves an LMMSE structure (model-based), providing more robustness unseen data, as well as good interpretability to the result. In this way, the solution adapts to the anatomy in every point of the image and noise properties at that particular location.

As an exemplary, non-limiting, embodiment of the disclosure, let us assume a Linear Minimum Mean Square Error (LMMSE) to denoise a two-material images after FPB, i.e., x=[x₁, x₂]. This is the solution to:

$${min}_{a} ‖ a - x ‖ ,$$

subject to a=Wx+b, where a=[a₁, a₂] contains the resulting denoised images, and W and b are the parameters of the linear denoising. Thus, the LMMSE solution is:

$$\hat{W} = {\sum }_{ax} {\sum }_{x}^{- 1} \hat{b} = a - \overline{W} x ,$$

where Σₓ is the covariance matrix of the noisy FBP result, x, and Σₐₓ the cross-covariance matrix between noisy and clean images. Here, we let W and b denote a general matrix and vector used in a linear transformation a=Wx+b whereas Ŵ and \hat{b} denote specific instances of this matrix and vector, obtained for example by processing spectral image data through a neural network.

Although finding Ŵ and \hat{b} may seem initially simple, several problems may be encountered. The first is the dimensionality of matrix Ŵ, which is unfeasible due to the very high dimensional images that we are dealing with. Therefore, the cross- and co-variance analysis will have to be restricted to relations between a limited number of pixels. The simplest case would be to only assume the diagonal of the cross- and co-variance matrices, which would be computationally simple but too simplistic, as well as a very biased approximation. Nevertheless, we will use this case as a starting point to our deep learning approach. The second problem is that these matrices, as well as the mean values @ and x are initially unknown and need to be estimated with a sufficient amount of observed data. We will use our training data to perform these estimates as sample cross- and co-variances and sample means.

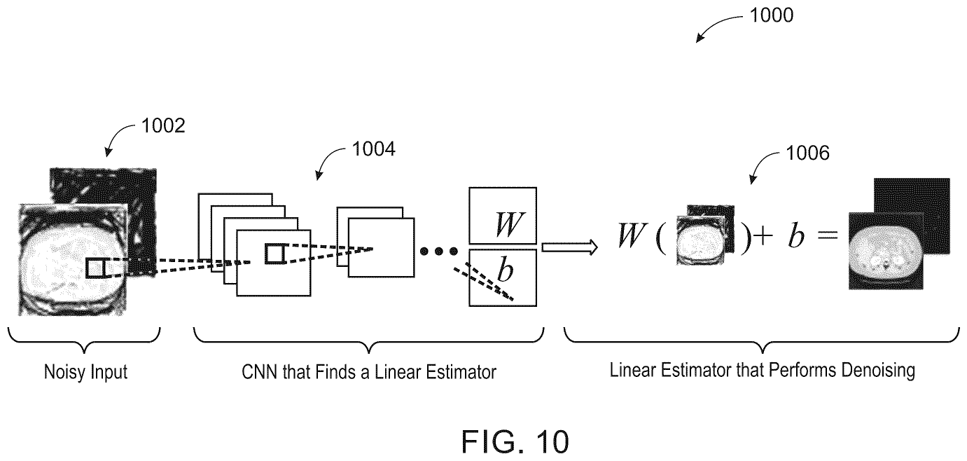

Let us explain how we consider a model-based deep learning in this scenario. We have a model-based solution (the LMMSE denoiser) that we need to enhance in order to obtain good estimates for W and b when we use only diagonal cross- and co-covariances. Therefore, we wish to preserve the mathematical structure (linear, fast) with a deep learning inference (to estimate the LMMSE parameters with a powerful statistical model-agnostic approach). Enforcing a problem structure, we aim for a neural network that needs few training samples, and is more robust to unseen datasets than typical “black-box” networks. Of course, this result is also expected to be much better than considering diagonal cross- and co-variances in a too simplified LMMSE. We have represented our deep learning proposed solution in FIG. 10.

One can give an additional interpretation for the results. The goal of the network is to obtain W and b instead of the denoised image. Therefore, if one wants to manipulate and understand the solution, instead of changing or accessing the millions of parameters inside a CNN, one could consider the parameters in W and b, which are considerably fewer and also more “interpretable” (in connection to an LMMSE).

The proposed deep learning approach requires a training database. To show a proof of concept of the proposed disclosure we have trained a learned LMMSE estimator with simulated photon counting data. We have simulated a set of 1200 cases, where the PCCT measurements are computed with an eight-bin silicon detector, and then a two-material decomposition and FBP are performed to obtain the material images. We have used the KiTS19 database, mostly composed of abdominal scans. 1000 samples are used to train and 200 to test. In order to evaluate the robustness of the techniques to adapt to unseen data, we have also simulated 200 extra scans from a different database (NSCLS), which also contains full body scans and thus more variability in anatomy than the training database.

In this example, PyTorch and one NVIDIA GPU Geforce RTX 2080 Ti GPU board have been used to train the neural networks. In order to perform a comparative study, we consider the following competing solution: (1) The original simplistic LMMSE, as described in previous section; and (2) A “black-box” CNN based on the UNet architecture.

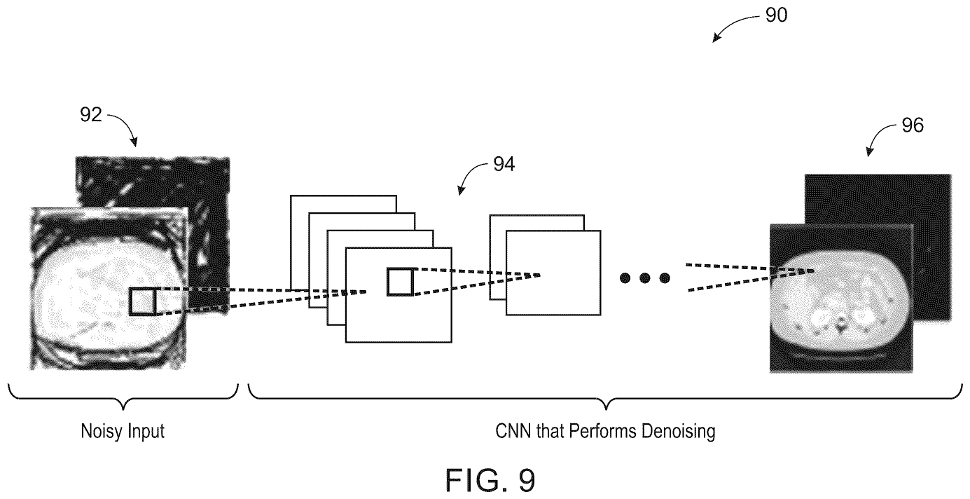

FIG. 9 shows a schematic representation of a conventional CNN-based denoising technique 90, in which a black-box CNN 94 maps directly from a noisy CT image 92 to the clean CT image 96.

FIG. 10 shows a schematic representation of the presented denoising technique 1000, in which a CNN 1004 accepts a noisy CT image 1002 as an input, the CNN finds a linear estimator 104, the CNN is used to map the linear model parameters W and b, and the linear estimator is used to perform denoising 1006 and generate a clean CT image. Therefore, a linear structure is enforced in the learning process.

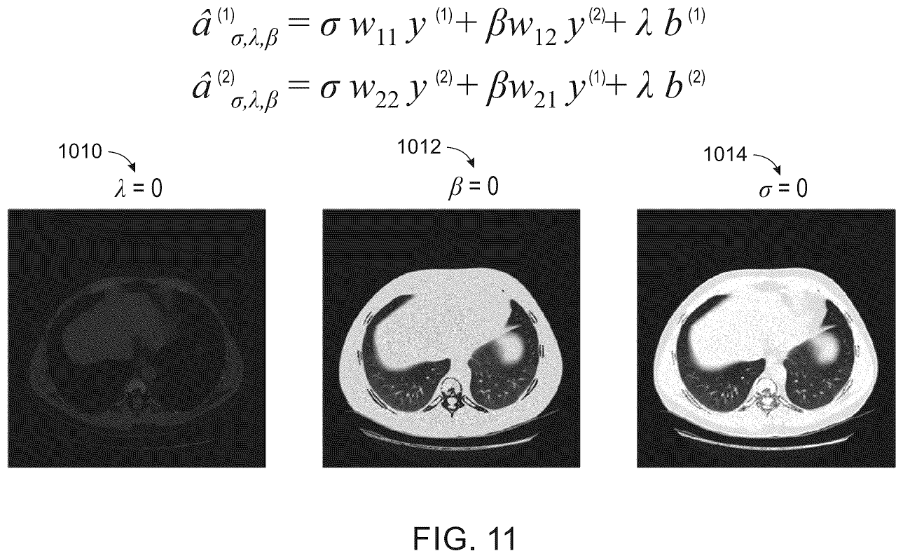

FIG. 11 shows an example of its interpretability: it is possible to manipulate the learned linear components W and b with only a few parameters. Three parameters, σ, β and λ are used to control the wᵢᵢ (“variance” of single material component of the linear model), wᵢⱼ (“cross-covariance” of opposite material component) and b (mean or “bias” of single material component). When the bias is zero (λ=0) 1010 an enhanced structure is shown in the result. If the “cross-covariance” wᵢⱼ is zero (β=0) 1012 the cross-contamination between materials may be less corrected. Furthermore, if the “variance” component is zero (σ=0) 1014 it can be the single material noise variance that is not reduced.

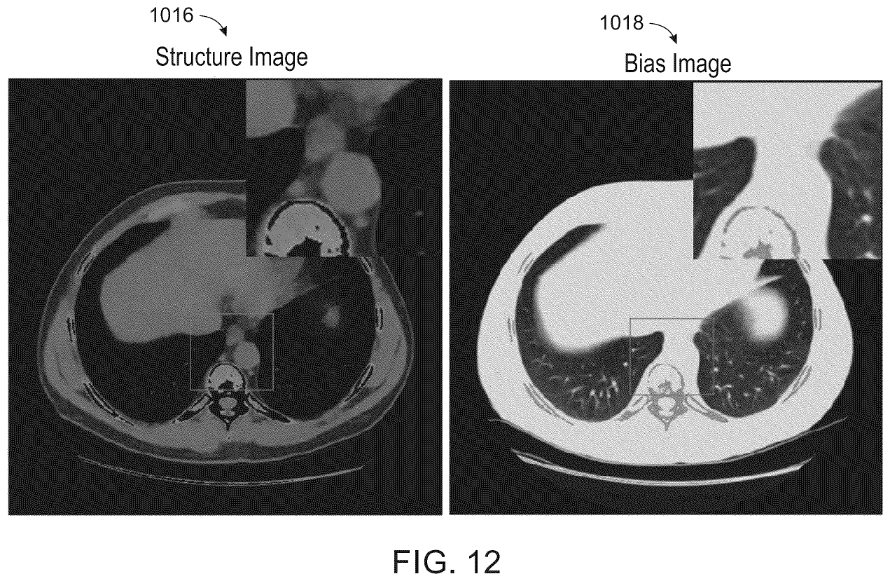

FIG. 12 shows an example of interpretation of what the two components in a=Wx+b provide to the result. First term (Wx) provides details about the structure of the anatomy (finer edges, local small structures) as shown in CT image 1016 (structure image), but does not give an accurate CT number that is expressed in Hounsfield Units (HU). The second independent term, b, generally corrects the HU values of the results (the mean, or bias) as shown in CT image 1018 (bias image) with very few details of the anatomy (very smoothed image).

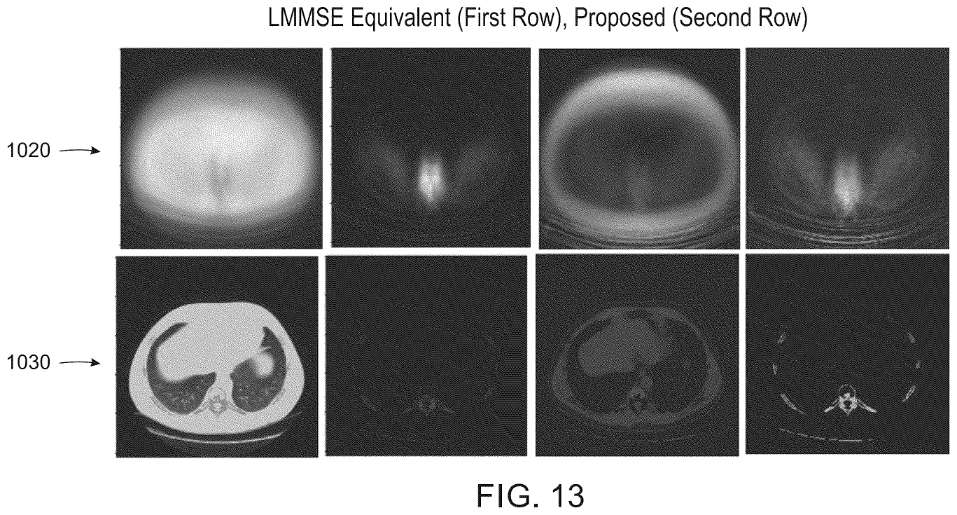

FIG. 13 shows an empirical demonstration of the parallelism of the technique and an LMMSE: first row 1020 represents the estimator components of a regular LMMSE for a particular phantom example, and second row 1030 their equivalent with our learned approach. One can see that, the LMMSE is not specific enough and the result is too blurry and does not represent accurately the denoised example. However, our approach is much more specific and accurate, but preserved similarities with the mentioned LMMSE. Hence, it can be interpreted as a DNN-enhanced LMMSE (which is the best linear estimator to minimize MSE).

The disclosure relates to a spectral or energy-resolved image data which consists of image data containing a at least two spectral components. In this context image data can be for example be two-dimensional, three-dimensional, or time-resolved and refer to either reconstructed images or an intermediate representation of image data such as a sinogram. The different spectral components can for example be synthetic monoenergetic images, wide-spectrum images acquired at different tube acceleration voltages or material-selective images, such as basis images. The different spectral components can also be a combination of the above.

The above description should be understood to be exemplary and non-limiting, and several variations of the described method can be envisioned. For example, several different architectures of the convolutional neural network are possible, such as UNet, ResNet or an unrolled iterative network, e.g., an unrolled gradient descent or unrolled primal-dual network. Furthermore, it may or may not be desirable to include batch normalization, skip connections and in the network training, and different pooling layers such as maximum pooling, average pooling or softmax pooling can be included in the network. Different loss functions can be minimized while training the network, such as L1 loss, L2 loss, perceptual loss and adversarial loss. Perceptual loss can be implemented with different discriminator networks, and different layers of such networks can be used in order to obtain different image characteristics.

The inventors have appreciated that it is impractical to let W be a full matrix and obtain all of its elements using the neural network, since this would require a neural network with on the order of 10¹² outputs. Therefore, it is desirable to impose some structure on the matrix, for example by letting W be a sparse matrix, i.e., a matrix with a small number of nonzero elements. For example, the matrix W may be a diagonal matrix in which case each pixel value in the set of image data will be multiplied by a value when the matrix is applied. Another option is to let W be block diagonal. For example, if the spectral data consists of N spectral components, W can for example consists of blocks of N×N elements along its diagonal, such that applying W to a vector causes values corresponding to the different spectral components in one particular pixel to be transformed by the N×N block to a new set of spectral components in the corresponding transformed set of spectral component images.

Another example is to let W act on each of the different spectral components separately, with the entries corresponding to cross-talk between different components being set to zero. Both in the case of W acting on each of the different spectral components separately and in the more general case of W including cross-component entries corresponding, can be taken to be those corresponding to a certain maximal distance in pixels between the input pixel and the output pixel. Alternatively, W can be represented as a transformation in the Fourier domain W=F⁻¹W_{F}F where F is a Fourier transformation operator and only elements of W_{F} corresponding to certain frequencies, such as low or high frequencies, are nonzero.

Other examples include letting W be an element of the range of a linear or nonlinear transformation, such as for example an artificial or convolutional neural network.

The vector b can also be chosen to be for example a full vector without any restrictions or a sparse vector where only certain elements are nonzero. In another exemplary embodiment of the disclosure, b can be restricted to the range of a linear or nonlinear transformation, for example an artificial or convolutional neural network, or as a linear combination of Fourier components b=F⁻¹b_{F} where by is a vector of Fourier components of b that can for example be restricted to contain high or low spatial frequencies.

In practice, imposing such restrictions on W and b can be done by letting a convolutional neural network output only those elements of W and b that should be nonzero and setting the other components to zero. In another embodiment of the disclosure, a convolutional neural network may generate a feature vector that is subsequently transformed into W and b, for example through a linear transformation or through an artificial or convolutional neural network.

For example, a number of Fourier components can be generated by way of a neural network and the transformed to form for example b, or one or more diagonals of W. In another embodiment of the disclosure, different components of b and/or W, or a feature vector related to b and/or W are given different weight in a loss function used to train a neural network to generate these components, or are penalized by a penalty term making it unlikely that these component will attain values of large magnitude. For example, the components of b and/or W can be regularized in such a way that high spatial frequencies are penalized, meaning that these components will contain predominantly low frequencies. In this way too large variations between the transformations applied to neighboring pixels can be avoided, making the denoising method more robust to differences in noise characteristics and image appearance compared to the training dataset. In another example, low frequencies can be penalized, providing a denoiser particularly suited for preserving fine details.

The inventors have appreciated that the linear structure of this denoiser can provide both explainability and tuneability. The learned LMMSE denoiser is similar in its mathematical structure to the conventional LMMSE denoiser that is based on a handcrafted noise model. By comparing the coefficients of the learned LMMSE denoiser to those of the conventional LMMSE denoiser, information about how the denoiser acts on images can be obtained. For example, such a comparison can show that the action of the learned LMMSE denoiser in a limited area of the image is similar to a conventional LMMSE denoiser built on certain models of signal and noise. This information can prove useful when seeking to analyze the image quality and robustness properties and improving the learned LMMSE denoiser, for example by adjusting the structure of b and/or W or the training parameters.

The structure of the linear LMMSE denoiser also provides tunability to the model. For example, individual entries or groups of entries in \hat{b} and/or Ŵ can be tweaked to obtain an image with desired properties. For example, entries of \hat{b} and/or Ŵ that control the pixel values in a particular region of the image can be adjusted to adapt image properties in a particular region of interest. In another example, the values of the diagonal of Ŵ, or values that belong to the diagonal blocks of a block-diagonal Ŵ, can be adjusted in order to attain specific image properties.

Such manipulation of coefficients can take place by multiplying a selected set of coefficients with a constant factor. Alternatively, it can take place by interpolating between Ŵ x+\hat{b} and the identity transformation that corresponds to setting W equal to the identity matrix and b to zero. In this way new learned linear transformation a=Ŵ′x+\hat{b}′ can be obtained, for which select components are more similar to the identity transformation compared to the previous transformation Ŵx+\hat{b}.

By way of example, the inventors have appreciated that Ŵ tends to be related to the structure of the image whereas b is related to the large-area bias. By changing the relative weight of Ŵ and \hat{b} it is therefore possible to obtain a desired trade-off between structure and bias. This can be achieved for example by multiplying Ŵ or selected elements of Ŵ with a scalar value, and multiplying \hat{b} or selected elements of \hat{b} by another scalar value. For example, \hat{b} can be multiplied by a value between 0 and 1 to enhance the representation of structures in the image while accepting a higher bias. In another example, Ŵ can be multiplied by a value between 0 and 1 to decrease image bias in situations where detailed structures are less important.

In another embodiment of the disclosure, the tunability is achieved by training a single neural network to generate a family of matrices Ŵ(t) and vectors \hat{b}(t) based on a tuning parameter 1, such that varying t gives images a=Wx+b with different characteristics. For example, images with different resolution or bias properties can be obtained. In another example, different t can give images with different noise textures. This can be achieved by using a training dataset where each training sample consists of one spectral input image dataset and a plurality of spectral output image datasets. The loss function used for training the neural network can then incorporate one term per output image dataset penalizing the difference between the network output for different values of t and each of the output image datasets.

In another example, t can be replaced by a plurality of tuning parameters allowing several different properties of the image to be tuned.

In yet another embodiment of the disclosure, tunability can be achieved in real time while displaying an image to the end user, allowing the user to adjust the image to obtain the desired image properties.

In an exemplary embodiment of the disclosure, the convolutional neural network is trained by minimizing a L1 loss function, a L2 loss function, a perceptual loss function, an adversarial loss function or a combination of these.

The goal of the network is to obtain W and b such that a=Wx+b for a=[a₁, a₂], the denoised images corresponding to the material images x=[x₁, x₂]. Though the example here is described for the case of two spectral components, this is a non-limiting example and the vectors a and x can in general have any number of components larger than or equal to two. This goal is achieved by training the network using a L2 loss function,

$${l}_{2} ( \hat{a} , a ) := {‖ \hat{a} , a ‖}_{2}^{2}$$

where â:=Ŵx+\hat{b} and Ŵ and \hat{b} are the output from the network. One could also use the L1 loss [symbol] ₁(â, a):=∥â−a∥₁. The L2 and L1 loss are pixel-wise loss functions that are known to cause over-smoothing and loss of fine-grained details that may be important to the perceptual quality and clinical usefulness of the resulting image.

One possible solution is to use a feature-based perceptual loss which, instead of comparing output and ground truth pixel-per-pixel, compares the feature representations corresponding to the output and ground truth. The feature representations are obtained by passing the target and output through a pretrained Convolutional Neural Network (CNN). For instance, VGG16/19 (CNN from the visual geometry group at the University of Oxford) are commonly used as feature extractor. The perceptual loss has been used in a variety of computer vision problems such as image denoising and super-resolution. Let ϕⱼ denote the j-th layer of a pretrained CNN, then the perceptual loss is defined as

$${l}_{perception} ( \hat{a} , a ) := {‖ {ϕ}_{j} ( \hat{a} ) - {ϕ}_{j} ( a ) ‖}_{2}^{2} .$$

Another possibility is to minimize some notion of distance between the distribution of the ground truth and output images. This can be achieved using an adversarial loss, based on Generative Adversarial Networks (GANs). In this setting, we pit the network against another CNN in a minimax game which, through successive improvements, will encourage the distribution of the output to be indistinguishable from that of the ground truth. This may prevent excessive denoising and over-smoothing associated with pixel-wise losses such as the L2 and L1 loss. Let [symbol] ₐ be the distribution of the ground truth material images, [symbol] ₓ the distribution of the noisy material images, and [symbol] _{â} be the distribution implicitly defined by â:=Ŵx+\hat{b} where Ŵ and \hat{b} are the output from the network G. Let the network which we are pitting G against be denoted D for discriminator. The job of the discriminator is to discriminate (classify) between real and generated output. The original version of GAN solves the following minimax game:

$${min}_{G} {max}_{D} {𝔼}_{a \sim {ℙ}_{a}} [ log ( D ( a ) ) ] + {𝔼}_{x \sim {ℙ}_{x}} [ log ( 1 - D ( G ( x ) ) ) ] .$$

For an optimal discriminator, the objective of the generator is tantamount to minimizing the Jensen-Shannon divergence between [symbol] ₐ and [symbol] _{â}. Though capable of producing amazing results, GANs are notoriously difficult to train. One version that mitigates the common issues of vanishing gradients and mode collapse is the Wasserstein GAN with gradient penalty (WGAN-GP). The WGAN-GP strives to minimize the Earth-mover or Wasserstein distance between [symbol] ₐ and [symbol] _{â} instead of the Jensen-Shannon divergence. The discriminator is now called a critic, which we denote C, as it now outputs any real number instead of a number in [0,1] and is therefore do no longer discriminate. The minimax game is:

$${min}_{C} {max}_{G} {𝔼}_{\hat{a} \sim {ℙ}_{\hat{a}}} [ C ( \hat{a} ) ] - {𝔼}_{a \sim {ℙ}_{a}} [ C ( a ) ] + {λ𝔼}_{\hat{a} \sim {ℙ}_{\hat{a}}} [ {( {‖ {∇}_{\hat{a}} C ( \hat{a} ) ‖}_{2} - 1 )}^{2} ] ,$$

where the final term is a gradient penalty that softly enforces that the critic is 1-Lipschitz continuous and [symbol] _{ã} is the distribution implicitly defined via ã:=ϵa+(1−ϵ)â for ϵ˜U[0,1]. This linear combination of a and â is used instead of checking the gradient everywhere, which would be intractable. The 1-Lipschitz continuity condition on the critic is necessary to get a tractable version of the Wasserstein distance. In contrast to a standard GAN, which takes a stochastic input to produce some realistic, but stochastic output, one can use this setup to train a learned LMMSE by sampling a pair of noisy material images x instead of a stochastic noise vector (as is done normally in a GAN). In addition, one can favorably combine the adversarial loss with a reconstruction loss such as the perceptual loss.

WGAN-GP is not necessarily the best performing GAN, however it is one of the most stable to train. Previous publications have demonstrated the stability of the WGAN-GP on several different tasks and datasets without experiencing the common issues of vanishing gradients and mode collapse.

To trade off advantages and disadvantages of these loss functions, one can consider a weighted sum of previously mention loss functions.

In an exemplary embodiment of the disclosure, the convolutional neural network is trained as part of a pair of cycle-consistent generative adversarial networks.

The data required for this disclosure is paired samples of noisy material images and their ground truth (low noise) counterparts. However, in many cases such paired datasets are not available. Instead, we might have a pile of noisy material images and a pile of denoised/low noise material images. To extend the learned LMMSE to unpaired data one can apply a so-called cycle-consistent GAN. The key insight that enables this is the cycle-consistent loss. The objective is to find a map from the source domain X to the target domain A. Let G:X→A, be the map which takes a pair of noisy material images x, pass them through our network and form denoised material images a=Wx+b. Using an adversarial loss, we can push the distribution induced by G(X) such that it is indistinguishable from that of A. However, this mapping is highly under-constrained and the space of possible mappings in huge. To reduce the space of possible mappings, one can consider the inverse mapping F: A→X and enforce cycle consistency via a cycle-consistent loss. Our mapping is the to be cycle-consistent if F(G(x))≈x∀x∈X and G(F(a))≈a∀a∈A. Cycle-consistency can be enforced via the cycle-consistent loss:

$${l}_{cycle} ( G , F ) := {𝔼}_{x \sim {ℙ}_{x}} ⌈ {‖ F ( G ( x ) ) - x ‖}_{1} ⌉ + {𝔼}_{a \sim {ℙ}_{a}} ⌈ {‖ G ( F ( a ) ) - a ‖}_{1} ⌉ .$$

This is combined with a GAN for the mapping G and the inverse mapping F each with their own discriminator D_{A} and D_{X}, respectively. Hence, we have the objectives:

$${l}_{GAN} ( G , {D}_{A} , X , A ) := {E}_{a - {ℙ}_{a}} [ log ( {D}_{A} ( α ) ) ] + {𝔼}_{x \sim {ℙ}_{x}} [ log ( 1 - {D}_{A} ( G ( x ) ) ) ]$$

and similarly for [symbol] _{GAN}(F, D_{X},A, X). Putting it all together leads to the minimax game:

$${min}_{G , F} {max}_{{D}_{A} , {D}_{X}} {l}_{GAN} ( G , {D}_{A} , X , A ) + {l}_{GAN} ( F , {D}_{X} , A , X ) + {λl}_{cycle} ( G , F ) .$$

As with the original GAN, this formulation will have issues with training stability. To circumvent this, the negative log likelihood loss is replaced by a L2 loss. In other words, the generator is trained to minimize [symbol] _{x˜} [symbol] _{ₓ}┌(D_{Y}(G(x))−1)²┐ and the discriminator to minimize [symbol] _{a˜} [symbol] _{ₐ}[(D_{Y}(a)−1)²]+ [symbol] _{x˜} [symbol] _{ₓ}┌D_{Y}(G(x))²┐. In addition, a history of outputs (e.g., 50) is used to update the discriminator.

The method proposed by the present inventors comprises the steps of: (1) Acquiring energy-resolved CT image data; (2) Processing the energy-resolved CT image data based on at least one convolutional neural network such that a matrix Ŵ and a vector \hat{b} is obtained; and (3) Form denoised energy-resolved CT image data a as Linear denoiser a=Ŵx+\hat{b} based on the matrix W, where x is a representation of spectral CT image data containing at least two spectral components.

In an exemplary embodiment of the disclosure, at least one element of Ŵ or \hat{b} is adjusted to improve a measure of image quality.

In an exemplary embodiment of the disclosure, the measure of image quality is a mean-squared error, structural similarity, bias, fidelity of fine details, numerical observer detectability, visual grading score or observer performance.

In an exemplary embodiment of the disclosure, the matrix Ŵ is a diagonal matrix.

In another exemplary embodiment of the disclosure, the matrix Ŵ is a block-diagonal matrix, with its nonzero off-diagonal entries corresponding to the cross-terms between spectral components in each pixel.

In another exemplary embodiment of the disclosure, the matrix Ŵ is a sparse matrix, with nonzero elements corresponding to pixels located near each other.

In an exemplary embodiment of the disclosure, the convolutional neural network has a ResNet architecture, UNet architecture, unrolled iterative architecture or a combination of these.

In an exemplary embodiment of the disclosure, the convolutional neural network is trained by minimizing an L1 loss function, an L2 loss function, a perceptual loss function, an adversarial loss function or a combination of these.

In an exemplary embodiment of the disclosure, the convolutional neural network is trained as a generator in a generative adversarial network.

In an exemplary embodiment of the disclosure, the convolutional neural network is trained as part of a pair of cycle-consistent generative adversarial networks.

In an exemplary embodiment of the disclosure, the energy-resolved image data x is a set of sinograms.

In another exemplary embodiment of the disclosure, the energy-resolved image data x is a set of reconstructed images.

In an exemplary embodiment of the disclosure, the different components of the energy-resolved image data x consists of monoenergetic image data at different monochromatic energies, or image data corresponding to different measured energy levels or energy bins, or different basis images.

In an exemplary embodiment of the disclosure, an end user is given the possibility to adjust components of the matrix Ŵ and vector \hat{b}.

In another exemplary embodiment of the disclosure, the convolutional neural network is trained on a dataset containing a plurality of low-noise image with different image characteristics for each high-noise image, and this neural network is trained to generate low-noise images with different characteristics for each setting of at least one tuning parameter.

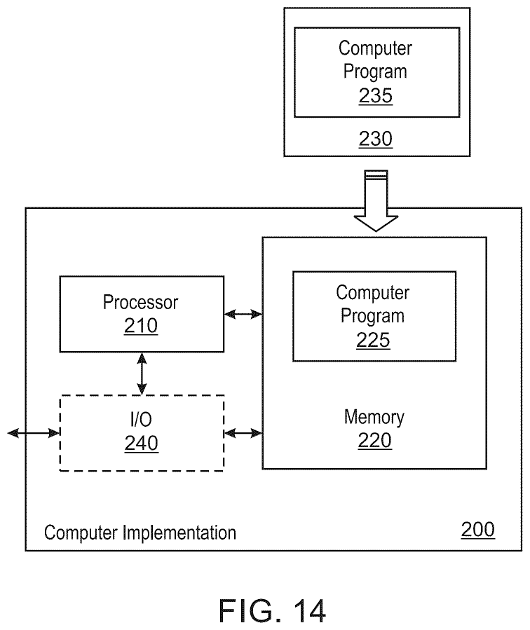

FIG. 14 is a schematic diagram illustrating an example of a computer implementation according to an embodiment. In this particular example, the system 200 comprises a processor 210 and a memory 220, the memory comprising instructions executable by the processor, whereby the processor is operative to perform the steps and/or actions described herein. The instructions are typically organized as a computer program 225; 235, which may be preconfigured in the memory 220 or downloaded from an external memory device 230. Optionally, the system 200 comprises an input/output interface 240 that may be interconnected to the processor(s) 210 and/or the memory 220 to enable input and/or output of relevant data such as input parameter(s) and/or resulting output parameter(s).

The term ‘processor’ should be interpreted in a general sense as any system or device capable of executing program code or computer program instructions to perform a particular processing, determining or computing task.

The processing circuitry including one or more processors is thus configured to perform, when executing the computer program, well-defined processing tasks such as those described herein.

The processing circuitry does not have to be dedicated to only execute the above-described steps, functions, procedure and/or blocks, but may also execute other tasks.

The proposed technology also provides a computer-program product comprising a computer-readable medium 220; 230 having stored thereon such a computer program.

By way of example, the software or computer program 225; 235 may be realized as a computer program product, which is normally carried or stored on a computer-readable medium 220; 230, in particular a non-volatile medium. The computer-readable medium may include one or more removable or non-removable memory devices including, but not limited to a Read-Only Memory (ROM), a Random Access Memory (RAM), a Compact Disc (CD), a Digital Versatile Disc (DVD), a Blu-ray disc, a Universal Serial Bus (USB) memory, a Hard Disk Drive (HDD) storage device, a flash memory, a magnetic tape, or any other conventional memory device. The computer program may thus be loaded into the operating memory of a computer or equivalent processing device for execution by the processing circuitry thereof.

The computer program residing in memory may thus be organized as appropriate function modules configured to perform, when executed by the processor, at least part of the steps and/or tasks described herein.

As mentioned, at least some of the steps, functions, procedures, and/or blocks described herein may be implemented in software such as a computer program for execution by suitable processing circuitry such as one or more processors or processing units.

Method flows may be regarded as a computer action flows, when performed by one or more processors. A corresponding device, system and/or apparatus may be defined as a group of function modules, where each step performed by the processor corresponds to a function module. In this case, the function modules are implemented as a computer program running on the processor. Hence, the device, system and/or apparatus may alternatively be defined as a group of function modules, where the function modules are implemented as a computer program running on at least one processor.

The computer program residing in memory may thus be organized as appropriate function modules configured to perform, when executed by the processor, at least part of the steps and/or tasks described herein.

Alternatively, it is possible to realize the modules predominantly by hardware modules, or alternatively by hardware. The extent of software versus hardware is purely implementation selection.

As used herein, an element or step recited in the singular and proceeded with the word “a” or “an” should be understood as not excluding plural of the elements or steps, unless such exclusion is explicitly stated. Furthermore, references to “one embodiment” of the present disclosure are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

Embodiments of the present disclosure shown in the drawings and described above are example embodiments only and are not intended to limit the scope of the appended claims, including any equivalents as included within the scope of the claims. It will be understood by those skilled in the art that various modifications, combinations, and changes may be made to the embodiments without departing from the present scope as defined by the appended claims. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

It is further noted that the inventive concepts relate to all possible combinations of features unless explicitly stated otherwise. In particular, different part solutions in the different embodiments can be combined in other configurations, where technically possible.

1. A method for denoising spectral CT image data, the method comprising:
determining a denoised linear estimation of spectral CT image data by maximizing or minimizing a first objective function, wherein at least one parameter of the denoised linear estimation is determined by at least one machine learning system.

2. The method according to claim 1, wherein determining the denoised linear estimation of spectral CT image data comprises:
receiving spectral CT image data;
processing the spectral CT image data based on the at least one machine learning system such that a matrix W and a vector b is obtained; and
forming denoised spectral CT image data a according to the linear estimation as per a=Wx+b, wherein x is a representation of spectral CT image data comprising at least two spectral components.

3. The method according to claim 2, wherein at least one of the matrix W and the vector b is adjustable for optimizing at least one image quality metric of the CT image data by maximizing or minimizing the first objective function.

4. The method according to claim 2, wherein the first objective function is at least one of mean-squared error, structural similarity, bias, fidelity of fine details, numerical observer detectability, visual grading score and observer performance.

5. The method according to claim 2, wherein the matrix W is a diagonal matrix.

6. The method according to claim 2, wherein the matrix W is a block diagonal matrix, and non-zero off-diagonal entries of the matrix W corresponds to cross-terms between the at least two spectral components in each pixel of the spectral CT image data.

7. The method according to claim 2, wherein the matrix W is a sparse matrix, and nonzero elements of the matrix W corresponds to pixels of the spectral CT image data located adjacent to each other.

8. The method according to claim 2, wherein the at least one machine learning system is trained by minimizing at least one of a L1 loss function, a L2 loss function, a perceptual loss function, and an adversarial loss function.

9. The method according to claim 2, wherein the spectral CT image data x comprises at least one of a set of sinograms and a set of reconstructed CT images.

10. The method according to claim 2, wherein the at least two spectral components of the spectral CT image data x comprises at least one of monoenergetic image data at different monochromatic energies, image data corresponding to different measured energy levels or energy bins, and different basis images.

11. The method according to claim 2, wherein at least one of the matrix W and the vector b of the denoised spectral CT image data a is adjusted by an end user.

12. The method according to claim 2, wherein the at least one machine learning system comprises at least one convolutional neural network (CNN).

13. The method according to claim 12, wherein the at least one convolutional neural network is trained on a dataset containing a plurality of low-noise images with different image characteristics for each high-noise image, and trained for generating low-noise images with different characteristics for each setting of at least one tuning parameter.

14. A CT imaging system comprising:
an X-ray source configured to emit X-rays;
an X-ray detector configured to generate spectral CT image data; and
a processor configured to:
determine a denoised linear estimation of the generated spectral CT image data based on maximizing or minimizing a first objective function;
wherein the processor is further configured to determine at least one parameter of the linear estimation by at least one machine learning system.

15. The CT imaging system according to claim 14, wherein the processor is configured to:
process the spectral CT image data based on the at least one machine learning system such that a matrix W and a vector b is obtained; and
form denoised spectral CT image data a according to the linear estimation as per a=Wx+b, wherein x is a representation of spectral CT image data containing at least two spectral components.

16. The CT imaging system according to claim 14, wherein at least one of the matrix W and the vector b is adjustable to enable optimization of at least one image quality metric of the CT image data based on maximizing or minimizing a second objective function.

17. The CT imaging system according to claim 14, wherein the at least one second objective function is at least one of mean-squared error, structural similarity, bias, fidelity of fine details, numerical observer detectability, visual grading score and observer performance.

18. The CT imaging system according to claim 14, wherein the matrix W is a diagonal matrix.

19. The CT imaging system according to claim 14, wherein the spectral CT image data comprises at least one of a set of sinograms and a set of reconstructed images.

20. The CT imaging system according to claim 14, wherein at least one of the matrix W and the vector b of the denoised spectral CT image data a is adjustable by an end user.
